(19) United States
(12) Patent Application Publication
(10) Pub. No.: US 2010/0199991 A1
(43) Pub. Date: Aug. 12, 2010

(54) VENTILATORY SUPPORT AND RESUSCITATION DEVICE AND ASSOCIATED METHOD

(75) Inventor: Michael J. Koledin, Charlotte, NC (US)

Correspondence Address: ERIC HANSCOM, 7395 PORTAGE WAY, CARLSBAD, CA, 92011, US

(73) Assignee: HARTWELL MEDICAL CORPORATION, Carlsbad, CA (US)

(21) Appl. No.: 12/701,495

(22) Filed: Feb. 5, 2010

Related U.S. Application Data
(60) Provisional application No. 61/150,621, filed on Feb. 6, 2009.

(51) Int. Cl. A61M 16/20; A61M 16/00; A62B 7/10

(52) U.S. Cl. 128/205.12; 128/204.18; 128/205.24

(57) ABSTRACT

A ventilatory support and resuscitation device for providing short-term ventilatory support, as well as constant flow, pressure cycled ventilatory support for breathing and non-breathing patients is disclosed. The device includes a chamber and at least one port in flow communication with the chamber. The apparatus also includes a flexible diaphragm member positioned within the chamber and configured to seal the port from flow communication with the chamber while seated adjacent to the port, and to flex in response to pressure applied thereto so as to allow flow communication between the port and the chamber while the diaphragm is flexing. A flow restrictor in flow communication with the chamber may also be included. The device may be coupled to a patient adapter, whereby a manometer, entrainment unit, and safety pop-off and entrainment valve may be coupled thereto.

### CROSS REFERENCE TO RELATED APPLICATIONS

This application is a non-provisional application of U.S. Provisional Patent Application No. 61/150,621 filed on Feb. 6, 2009, the entirety of which is hereby incorporated by reference.

### STATEMENT REGARDING FEDERALLY SPONSORED RESEARCH OR DEVELOPMENT

This invention was not federally sponsored.

INVENTOR: Michael J. Koledin

### BACKGROUND OF THE INVENTION

### Field of the Invention

The present invention relates to a ventilatory support and resuscitation device and, in particular, to a device, or apparatus, for providing short-term ventilatory support, as well as constant flow, pressure cycled ventilatory support for breathing and non-breathing patients.

Short-term ventilatory support is often needed for patients suffering from illness or injury. Both manual and automated devices have been developed to aid the patient in breathing by delivering a gas to the patient at various flow rates, pressures, volumes, and/or times. For example, automatic pressure-cycled devices may be used, which operate solely by pressure during inhalation and exhalation, such that the volume of gas delivered and the time between cycles do not affect the ventilatory cycle. Typically, ventilatory support devices are monitored by a trained operator and may be used with breathing and non-breathing patients. However, some ventilatory support devices may be prone to occlusion (e.g., by vomit) and/or mechanical problems that lead to interruption of the ventilatory cycle. In addition, some ventilatory support devices may be expensive, may be subject to misuse, and/or may subject the patient to potential injury.

Therefore, there exists a need for an improved device that provides ventilatory and resuscitation to the patient for short-term ventilatory support. In addition, there exists a need for a device that is reliable, safe, inexpensive, and requires minimal supervision.

### SUMMARY OF THE INVENTION

Embodiments of the present invention may provide improvements over the prior art by, among other things, providing apparatus and methods for ventilatory support and resuscitation for a patient. According to one embodiment, an apparatus includes a chamber and at least one port in flow communication with the chamber. The apparatus also includes a flexible diaphragm member positioned within the chamber and configured to seal the port from flow communication with the chamber while seated adjacent to the port, and to flex in response to pressure applied thereto so as to allow flow communication between the port and the chamber while the diaphragm is flexing.

According to aspects of the apparatus, the diaphragm includes an inner region and an outer region, wherein the inner region is configured to seal the port and the outer region is configured to flex in response to pressure applied to the inner region. The diaphragm also includes an outer edge, wherein the outer edge is configured to frictionally engage the chamber and remain stationary while the outer region is flexing. The inner region may be a more rigid material than the outer region, and the outer region may include a plurality of annular ridges, wherein the outer region is configured to flex along the annular ridges. The annular ridges may be configured to flatten when the outer region is flexing. In addition, the diaphragm may have a larger cross-sectional area than the port. The apparatus may further include a resilient member configured to apply a biasing force against the diaphragm so as to bias the diaphragm adjacent to the port, wherein the diaphragm is configured to flex so as to overcome the biasing force in response to pressure applied thereto. The apparatus may also include a dial for selectively adjusting the pressure required to overcome the biasing force of the resilient member. For example, a pressure required to overcome a biasing force of the resilient member may be about 3-5 cm H₂O when the diaphragm is adjacent to the port.

According to additional aspects of the apparatus, the apparatus includes a flow restrictor in flow communication with the chamber, wherein the flow restrictor is configured to be in flow communication with the port when the diaphragm is flexing. The flow restrictor may have a proximal end and a distal end and an orifice extending through and between the proximal and distal ends for facilitating flow communication between the chamber and the environment. Moreover, the apparatus may employ a dial for selectively adjusting a flow rate through the flow restrictor.

Further aspects of the apparatus include a patient adapter coupled to the port and in flow communication therewith. The patient adapter may also include an inlet port configured to couple with tubing supplying a gas. The patient adapter may also include a patient connection port configured to couple with an endotracheal tube or a mask. According to one aspect, the apparatus includes a manometer coupled to the patient connection port. The patient adapter may further include at least one entrainment valve configured to allow additional gas flow to the patient in response to inhalation by the patient but to occlude gas from escaping to the environment in response to exhalation by the patient. The patient adapter may include a plurality of entrainment valves, wherein one of the entrainment valves comprises a safety pop-off valve configured to open in response to a predetermined pressure and allow gas to escape to the environment. Also, the apparatus may include a cover surrounding the safety pop-off valve that is configured to prevent occlusion thereof.

Another embodiment of the present invention is directed to a method for providing ventilatory support and resuscitation for a patient. The method includes providing an apparatus as described above and coupling the apparatus to a patient such that the patient and apparatus are in flow communication with one another and such that the diaphragm is configured to flex in response to pressure applied thereto resulting from exhalation by the patient so as to allow flow communication between the port and the chamber while the diaphragm is flexing. Aspects of the method include selectively adjusting the pressure required to flex the diaphragm and/or selectively adjusting the flow rate of gas exiting the chamber. The method may further include coupling a patient adapter to the port, wherein the patient adapter is configured to receive a gas source and deliver the gas source to the patient. The method may also include coupling the patient adapter to a gas source and selectively adjusting a flow rate of the gas source.

An additional embodiment of the present invention is directed to an apparatus for providing ventilatory support and resuscitation for a patient. The apparatus includes a chamber and at least one port in flow communication with the chamber. The apparatus also includes a flow restrictor in flow communication with the chamber and having a proximal end and a distal end, wherein the flow restrictor comprises an orifice extending through and between the proximal and distal ends for facilitating flow communication between the chamber and the environment. The apparatus further includes a valve positioned within the chamber and configured to seal the port from flow communication with the chamber while seated adjacent to the port and to open in response to pressure applied thereto so as to allow flow communication between the port, the chamber, and the flow restrictor while the valve is open. The flow restrictor may include a plurality of longitudinally extending slits in flow communication with the orifice.

Another embodiment of the present invention is directed to a patient adapter configured to be coupled with an apparatus for providing ventilatory support and resuscitation for a patient. The patient adapter includes an inlet port configured to couple with a tubing supplying a gas and a patient connection port configured to deliver gas to the patient. The patient adapter further includes at least one entrainment valve configured to allow additional gas flow to the patient in response to inhalation by the patient but occlude gas from escaping to the environment in response to exhalation by the patient, wherein the at least one entrainment valve comprises a safety pop-off valve configured to open in response to a predetermined pressure and allow gas to escape to the environment. The patient adapter may include a cover surrounding the safety pop-off valve and configured to prevent occlusion thereof. In addition, the patient adapter may include a plurality of entrainment valves, wherein each entrainment valve is configured to allow additional gas flow to the patient in response to inhalation by the patient but occlude gas from escaping to the environment in response to exhalation by the patient. The patient adapter may include an entrainment unit coupled to an inlet port whereby tubing supplying gas to a patient is connected thereto. The entrainment unit, when open, is configured to allow ambient air to enter the inlet port while also maintaining the requisite pressure for operating the apparatus.

An additional embodiment of the present invention is directed to a manometer configured to be coupled with an apparatus for providing ventilatory support and resuscitation for a patient. The manometer may be electronic and include audible and/or visual alarms for signaling various events, such as when a predetermined pressure and/or rate is reached. The manometer may be coupled to the apparatus through a patient adapter or between a patient adapter and a port of the apparatus, whereby the manometer couples the patient adapter to the apparatus.

A further embodiment of the present invention is directed to a filtration unit with an optional quantifiable ETCO₂ colorimetry unit coupled with an apparatus for providing ventilatory support and resuscitation for a patient. The filtration unit may incorporate a high efficiency particulate absorbing (NEPA) filter that filters exhaled air from the patient before it reaches the apparatus. After exhaled air passes through the HEPA filter, whereby 99.9% of bacteria is removed, the exhaled air can pass through a quantifiable ETCO₂ colorimetry unit. The ETCO₂ colorimetry unit includes color metric paper that indicates CO₂ levels in the exhaled air. The filtration unit may also serve to protect a manometer coupled between the filtration unit and apparatus, whereby the manometer may be reused without requiring separate disinfecting.

It is a principal object of the invention to provide a ventilatory support resuscitation device to a patient for short-term ventilatory support.

It is another object of the invention to provide a ventilatory support and resuscitation device that is reliable, safe, inexpensive, and requires minimal supervision.

It is a further object of this invention to provide a method for providing ventilatory support and resuscitation for a patient.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

FIG. 1 is a perspective view of an apparatus for providing ventilatory support and resuscitation for a patient according to one embodiment of the present invention;

FIG. 2 is a perspective view of a modulator according to an embodiment of the present invention;

FIG. 3 is an exploded perspective view of the modulator shown in FIG. 2;

FIG. 4 is a perspective view of a flow restrictor according to one embodiment of the present invention;

FIG. 5 is a perspective view of a flexible diaphragm member according to an embodiment of the present invention;

FIG. 6 is an elevation view of the flexible diaphragm member shown in FIG. 5;

FIG. 7 is a cross-sectional view of a modulator and a valve in a closed position according to one embodiment of the present invention;

FIG. 8 is a cross-sectional view of the modulator and the valve shown in FIG. 7 in an open position;

FIG. 9 is an end view of a pressure dial according to one embodiment of the present invention;

FIG. 10 is a perspective view of a patient adapter according to an embodiment of the present invention;

FIG. 11 is an exploded perspective view of the patient adapter shown in FIG. 10;

FIGS. 12A and 12B are side views of an entrainment unit in a closed position and an open position, respectively, according to one embodiment of the present invention;

FIG. 13 is an exploded view of a safety pop-off valve according to an additional embodiment of the present invention;

FIG. 14 is an elevation view of a manometer according to one embodiment of the present invention;

FIG. 15 is a side view of the manometer shown in FIG. 14 connected to a coupling according to an embodiment of the present invention;

FIG. 16 is an exploded perspective view of the manometer shown in FIG. 16;

FIG. 17 is a side view of a filtration unit according to one embodiment of the present invention; and

FIG. 18 is a cross-sectional view of the filtration unit shown in FIG. 17.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

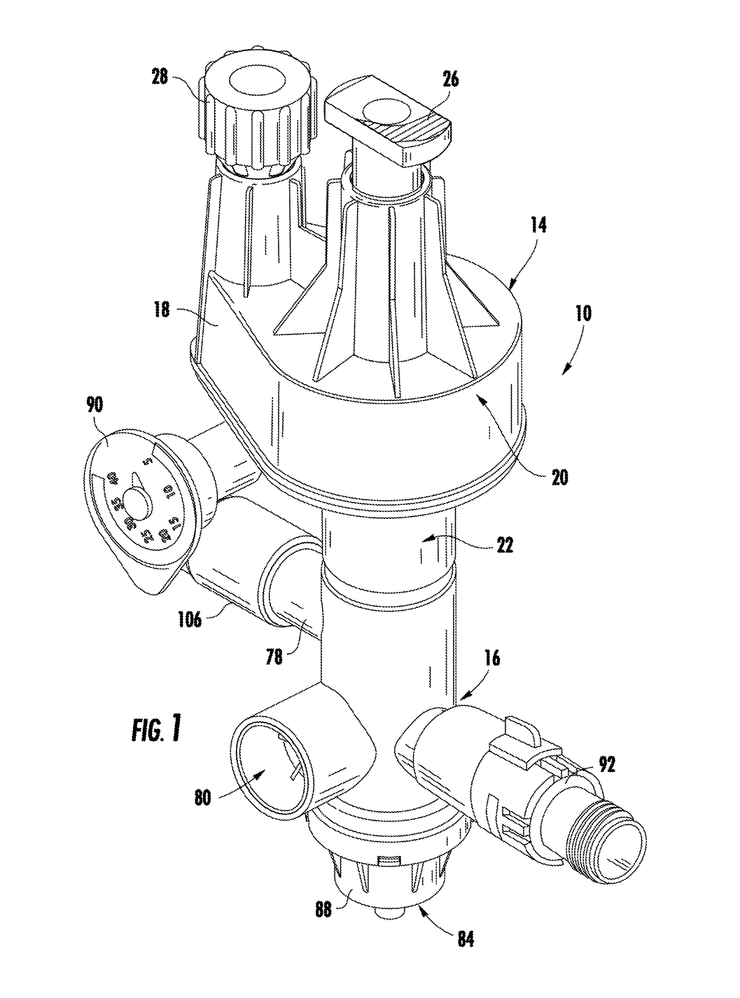

According to one embodiment of the present invention and with reference to FIG. 1, an apparatus 10 for providing ventilatory support and resuscitation for a patient is shown. The apparatus generally includes a modulator 14 and a patient adapter 16 for providing short-term ventilatory support, as well as constant flow, pressure cycled ventilatory support for breathing and non-breathing patients. The apparatus 10 is an automated device requiring only pressure for providing proper tidal volume, airway pressure, and respiratory rate to the patient. As explained in greater detail below, the modulator 14 includes a valve 20 that is configured to open and close in response to pressure during inhalation and exhalation. In addition and as also described below, the patient adapter 16 includes connections for a gas source and a patient, as well as various features for ensuring the patient's safety.

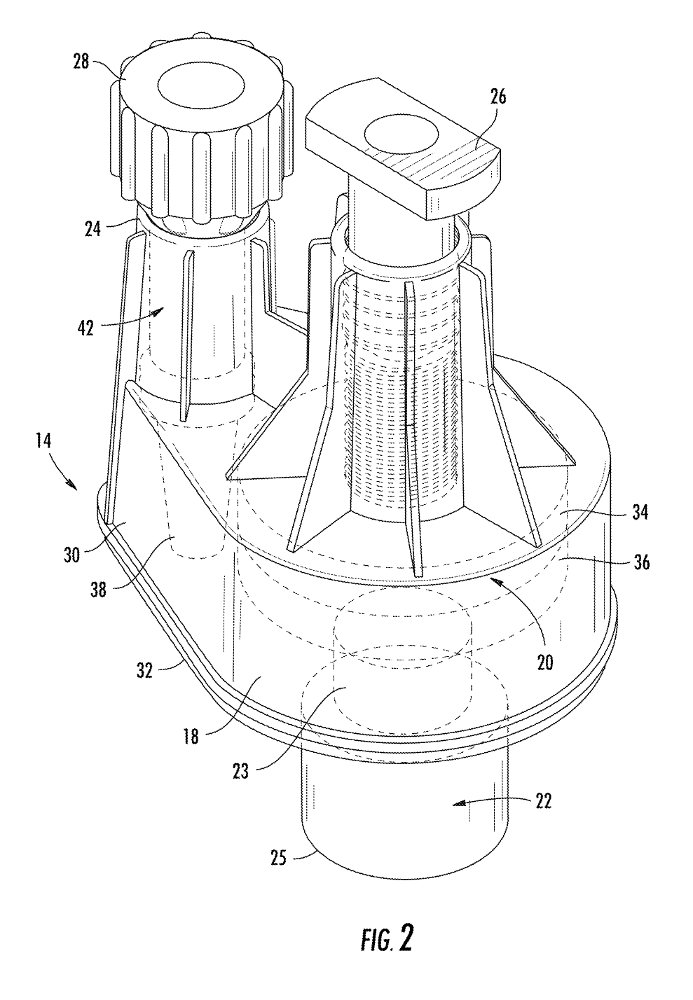

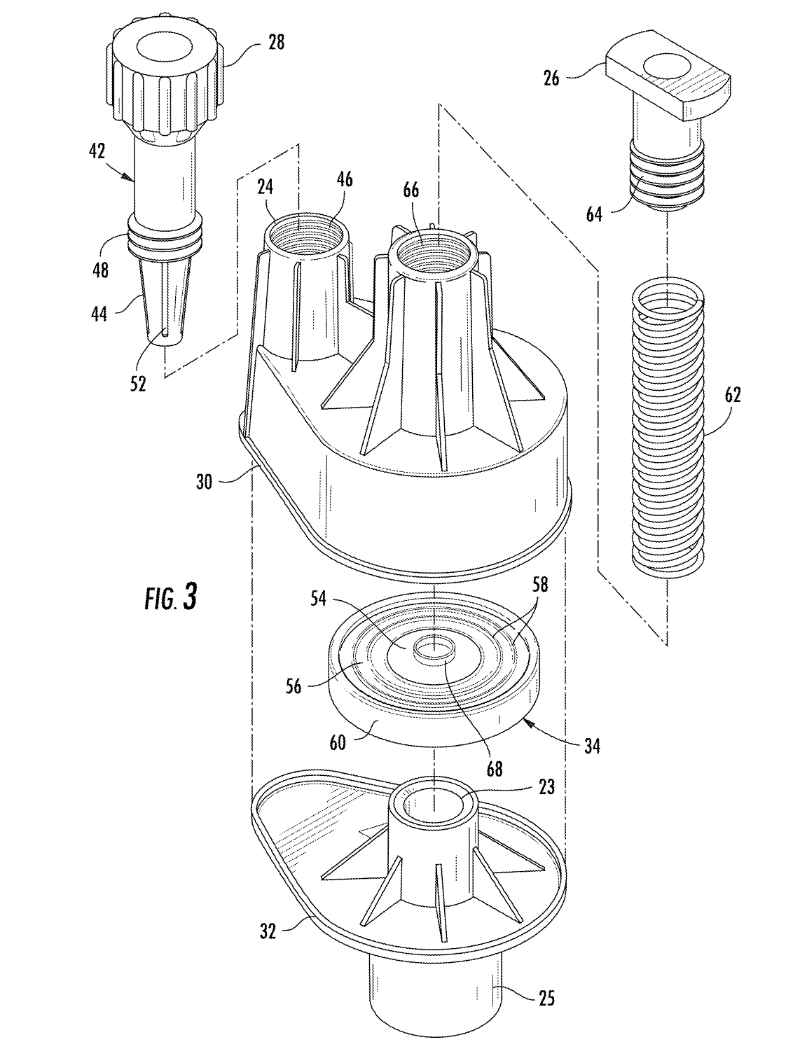

The modulator 14 generally includes a chamber 18, a valve 20, an inlet port 22, a flow restrictor port 24, a pressure dial 26, and a flow restrictor 42, as shown in FIGS. 2 and 3. The chamber 18 is formed by a housing having an upper portion 30 and a lower portion 32 coupled together. The chamber 18 may have a generally “tear drop” shape as shown in FIGS. 2 and 3, although it is understood that other shapes are possible such as oval or round. When there is no obstruction in the inlet port 22 for gas flowing therethrough and into the chamber 18, the inlet port and the flow restrictor port 24 are in fluid communication with one another. The lower portion 32 of the housing includes the inlet port 22 having an inner end 23 extending within the chamber 18 and an outer end 25 extending externally of the housing for coupling with the patient adapter 16. The diameter of the inlet port 22 may gradually reduce from external the housing to within the chamber 18. The upper 30 and lower 32 portions may be secured together with an adhesive, fasteners, a snap fit, or the like.

The flow restrictor port 24 may have a tapered end 38 that includes an opening defined therethough for allowing gas to flow between the chamber 18 and the external environment. The flow restrictor port 24 is configured to receive a flow restrictor 42 for selectively adjusting the flow rate of gas therethrough. In particular, the flow restrictor 42 includes a corresponding tapered end 44 that is configured to mate with the tapered end 38 of the flow restrictor port 24. The flow restrictor port 24 and flow restrictor 42 include internal 46 and external 48 mating threads. Thus, rotation of the flow rate dial 28 causes the flow restrictor 42 to rotate with respect to the flow restrictor port 24, which results in adjustment of the relative distance between the tapered ends 38, 44 and thereby adjustment of the rate of gas therethough. For example, threading the flow restrictor 42 into the flow restrictor port 24 results in moving the tapered ends 38, 44 closer together thereby reducing the flow rate through the flow restrictor, while threading the flow restrictor out of the flow restrictor port results in increasing the flow rate through the flow restrictor.

Moreover, as shown in FIG. 4, the flow restrictor 42 includes an orifice 50 extending through and between its proximal and distal ends, which allows gas within the chamber 18 to be constantly exiting, including when the flow restrictor 42 is bottomed out by turning the flow rate dial 28 clockwise completely within the flow restrictor port 24. Thus, the orifice 50 may allow for finer incremental adjustment at lower flow rates. The flow restrictor 42 may also include a plurality of longitudinal slits 52 extending between the distal end of the flow restrictor and the external threads 48 such that small rotational adjustments of the flow restrictor allow for a greater adjustment of the flow rate of gas escaping through the flow restrictor. The size of the orifice 50 may have any desired diameter to achieve a desired range of adjustment of flow rate. According to one embodiment, the orifice 50 may be variable in diameter. For example, the interior of the flow restrictor 42 may be threaded and configured to receive a corresponding threaded valve therein similar to a needle valve such that the orifice diameter is variable.

As alluded to above, the modulator 14 includes a valve 20 located within the chamber 18. The valve 20 includes a diaphragm 34 that is positioned within the chamber 18 and is configured to open and close in response to pressure. In particular, the diaphragm 34 is positioned within a guide 36 and is configured to abut the inlet port 22 in a closed position. As shown in FIGS. 2 and 3, both the diaphragm 34 and the guide 36 may have a circular shape, although other shapes could be employed according to additional aspects of the present invention (e.g., elliptical). FIG. 2 illustrates that the depth of the guide 36 extends partially within the chamber 18, such as about half of the height of the chamber, and that the thickness of the diaphragm 34 is less than the depth of the guide. In addition, the diaphragm 34 typically has a larger cross-sectional area than the inlet port 22.

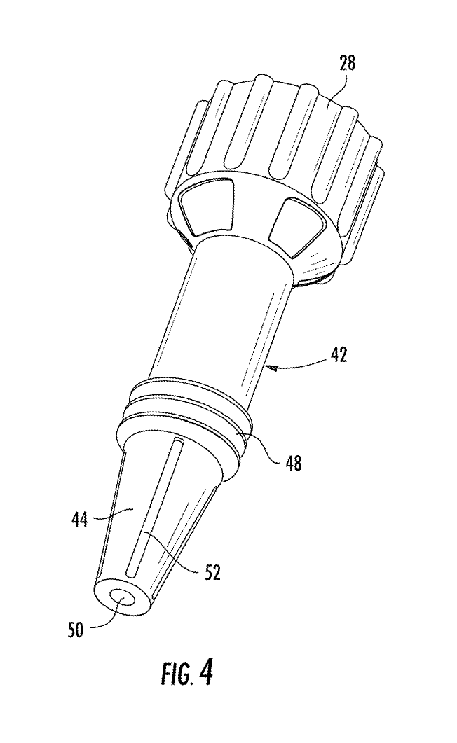

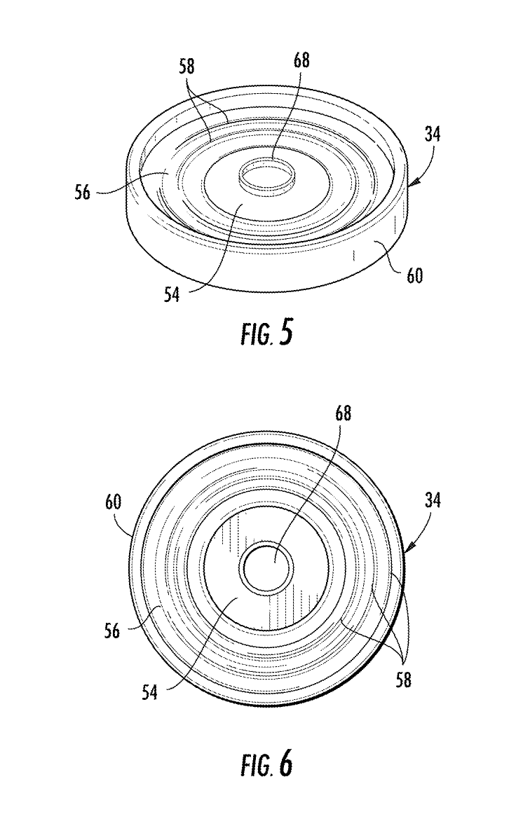

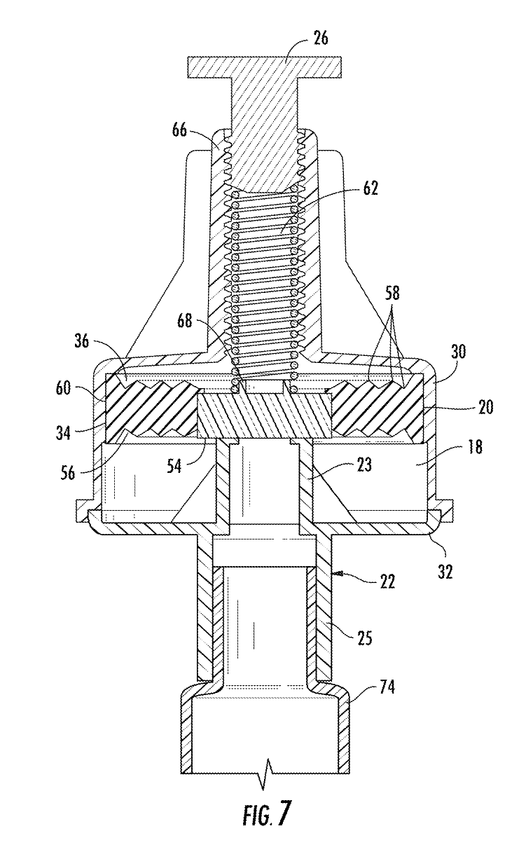

Referring to FIGS. 3, 5, and 6, the diaphragm 34 includes an inner region 54 and outer region 56, wherein the outer region is a flexible material. The inner region 54 is formed of a less flexible material than the outer region 56 (or even an inflexible material) and is configured to overlie the inner end 23 of the inlet port 22 and seal the port. Moreover, the outer region 56 is flexible such that it is capable of flexing in response to pressure applied to the inner region 54, such that the inner region is lifted off of the inner end 23 and gas is allowed to flow within the chamber 18. The outer region 56 may comprise a plurality of annular ridges 58 such that the outer region may flex along such ridges (see FIG. 7). The ridges 58 are configured to flatten out when the diaphragm 34 is flexing, which aids in keeping the diaphragm centered (see FIG. 8). When positioned within the guide 36, the outer edge 60 of the diaphragm 34 is frictionally engaged with the guide such that the outer edge of the diaphragm remains stationary whether the diaphragm is flexed or not flexed. As an alternative or in addition to the frictional engagement of the diaphragm and the guide, the diaphragm may be glued in place. Because the diaphragm 34 does not move axially within the guide 36, there is no concern that the diaphragm will become stuck or dislodged, which could be caused by moisture or fluid within the chamber (e.g., due to vomiting). According to one embodiment, the diaphragm 34 comprises polymeric materials. For example, the inner region 54 may be a polypropylene material, while the outer region 56 may be a elastomeric allow (TPV), such as modified polyphenylene oxide, silicone, Santoprene, or polytetrafluoroethylene material. Preferably, the inner region 54 is heat molded to the outer region 56 as glue cannot be used to bind the two regions together.

Opposite the diaphragm 34 of the inner end 23 is a resilient member 62, such as a spring or other biasing member. The resilient member 62 is configured to apply a biasing force against the diaphragm 34 so as to bias the diaphragm towards the inner end 23. The diaphragm 34 is configured to flex so as to overcome the biasing force in response to pressure applied thereto from gas entering the inlet port 22. The modulator 14 includes a pressure dial 26 that includes male threads 64 for engaging a female threaded conduit 66 that is in fluid communication with the chamber 18. Thus, as shown in FIGS. 3, 7, and 8, the threaded conduit 66 is configured to receive the resilient member 62, and the resilient member is configured to be positioned between the diaphragm 34 and the pressure dial 26. Threading the pressure dial 26 into or out of the threaded conduit 66 increases or decreases, respectively, the biasing force applied by the resilient member 62 on the diaphragm, thereby increasing or decreasing the pressure applied to the diaphragm 34 that is required to overcome the biasing force of the resilient member. When sufficient pressure is applied to the diaphragm 34 to overcome the biasing force of the resilient member 62, the diaphragm is configured to flex and thereby allow air entering the inlet port 22 to enter the chamber 18. Thus, there is sufficient head space between the diaphragm 34 and the distal end of the threaded conduit 66 in order to allow the diaphragm to flex and allow air to flow into the chamber 18.

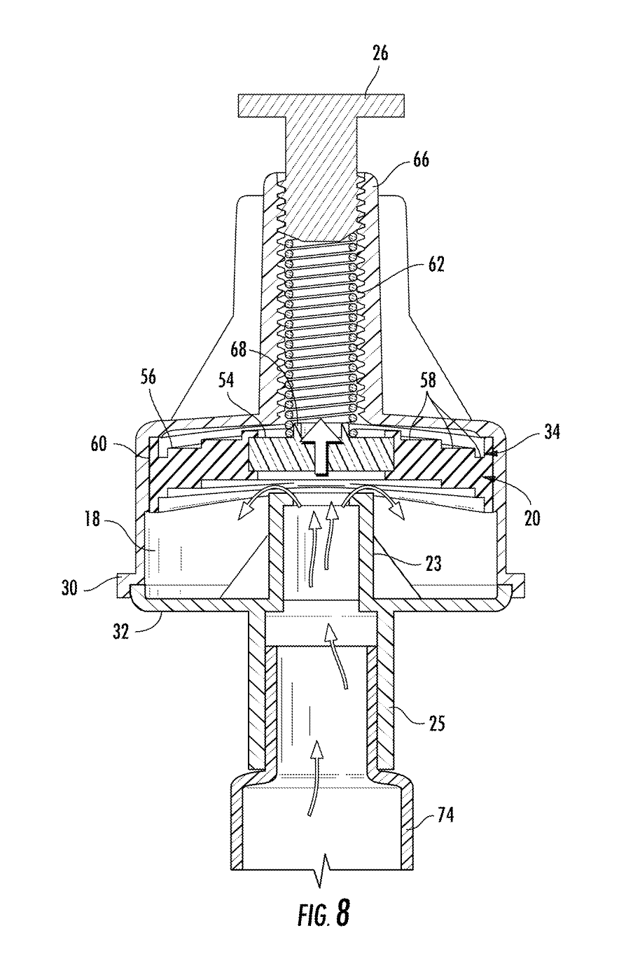

In order to facilitate proper positioning of the resilient member 62 when being biased, the diaphragm 34 may include a raised boss 68 that is configured to be encircled by one end of the resilient member, and the pressure dial 26 may include a recessed opening 70 and a pin 72 that are configured to receive the opposite end of the resilient member (see FIGS. 3 and 7-9). Thus, the diaphragm 34 and pressure dial 26 are capable of aiding in centering the resilient member 62 within the threaded conduit 66 and ensuring that a biasing force is applied generally in the center of the inner portion 54 of the diaphragm. The inner portion 54 and outer portion 56 may be dyed various colors, either the same or different colors. According to one embodiment of the present invention, a pressure required to overcome the biasing force of the resilient member 62 is about 3-5 cm H₂O when the diaphragm 34 is positioned adjacent to the inlet port 22.

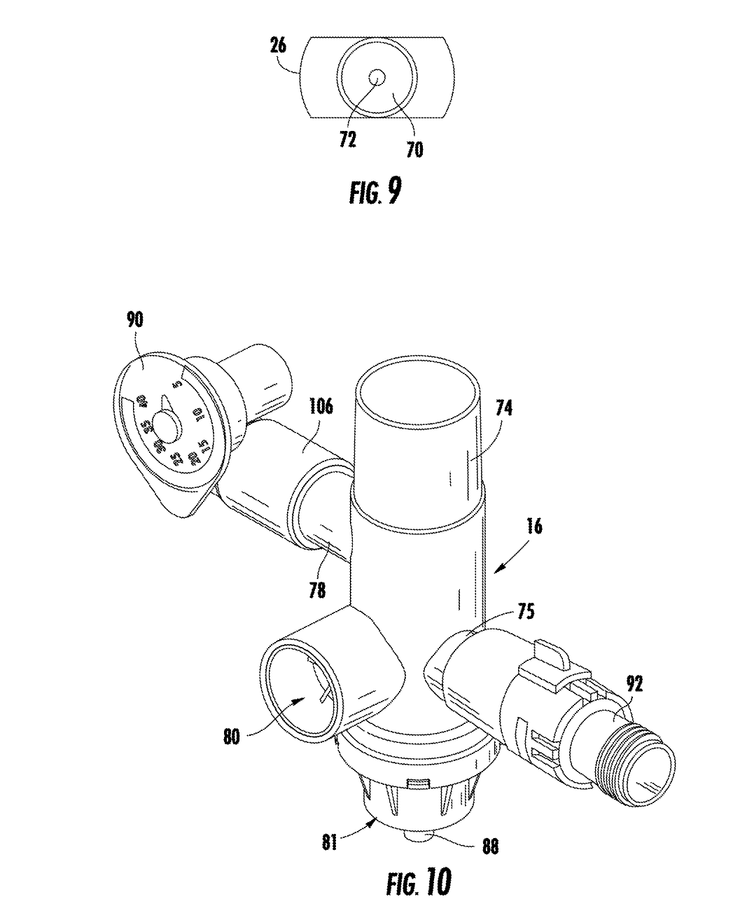

The patient adapter 16 is configured to be coupled to the modulator 14 and to the patient, as well as provide safeguards for the patient's safety. In particular, FIG. 10 shows that the patient adapter 16 generally includes an outlet port 74 configured to couple with the inlet port 22 of the modulator 14 such that the modulator and patient adapter may be in flow communication with one another. The patient adapter 16 also includes an inlet port 75 configured to couple with a tubing supplying a gas and a patient connection port 78 configured to couple with an endotracheal tube, a mask, or like device for facilitating the exchange of gas to and from the patient.

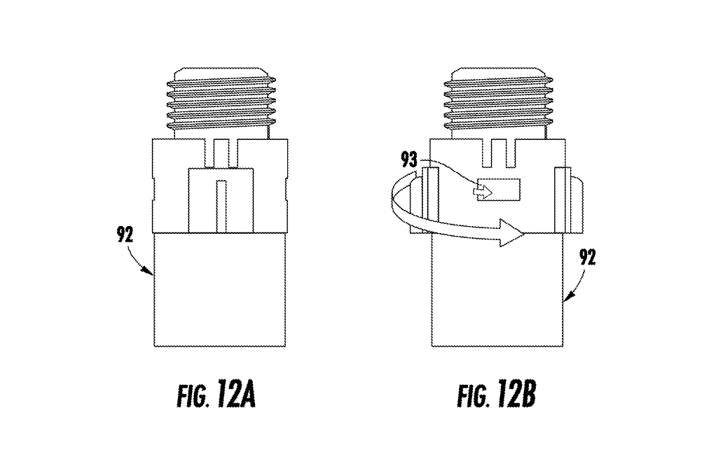

The inlet port 75 may receive pure O₂ or a combination of O₂ and ambient air. The patient may require pure O₂ until resuscitated and once the patient is resuscitated, the amount of O₂ may be reduced. In order to adjust the amount of O₂ delivered through the inlet port 75, an entrainment unit 92 may be coupled thereto. As shown in FIGS. 12A and 12B, the entrainment unit 92 is configured to rotate from a position that does not allow ambient air to enter the inlet port 75 to a position that allows ambient air to enter through opening 93. By allowing ambient air to enter the inlet port 75 through the opening 93, the amount of O₂ supplied may be reduced (e.g., 6 L of O₂ when the entrainment unit is open rather than 15 L O₂ when the entrainment unit is not open). The entrainment unit 92 may include one or more holes for allowing entrainment but also maintaining the requisite pressure for operating the apparatus 10.

To maintain a requisite pressure for operating the apparatus 10, the entrainment unit 92 should properly mate with the inlet port 75. The entrainment unit 92 may include a conical section 151 that acts as a nozzle to direct the flow of O₂ into the inlet port 75. The conical section 151 increases the speed of O2 as it exits the entrainment unit 92 thereby drawing in ambient air through the opening 93 when the entrainment unit is open. Likewise, the inlet port 75 should include an accepting nozzle 150 that allows for the efficient flow of O₂ from the conical section as well as ambient air from the opening 93, if the entrainment unit 92 is open. This configuration also helps to occlude the inlet port 75 when the patient is exhaling, thereby preventing gas from escaping to the environment while in operation. However, if an entrainment unit 92 is not used to connect an O₂ supply to inlet port 75, then it may be preferable to modify the design of the accepting nozzle 150 from the inlet port 75 to allow for the free flow of O₂ through the inlet port 75. The accepting nozzle 150 may or may not extend into the main cavity of the patient adapter 16.

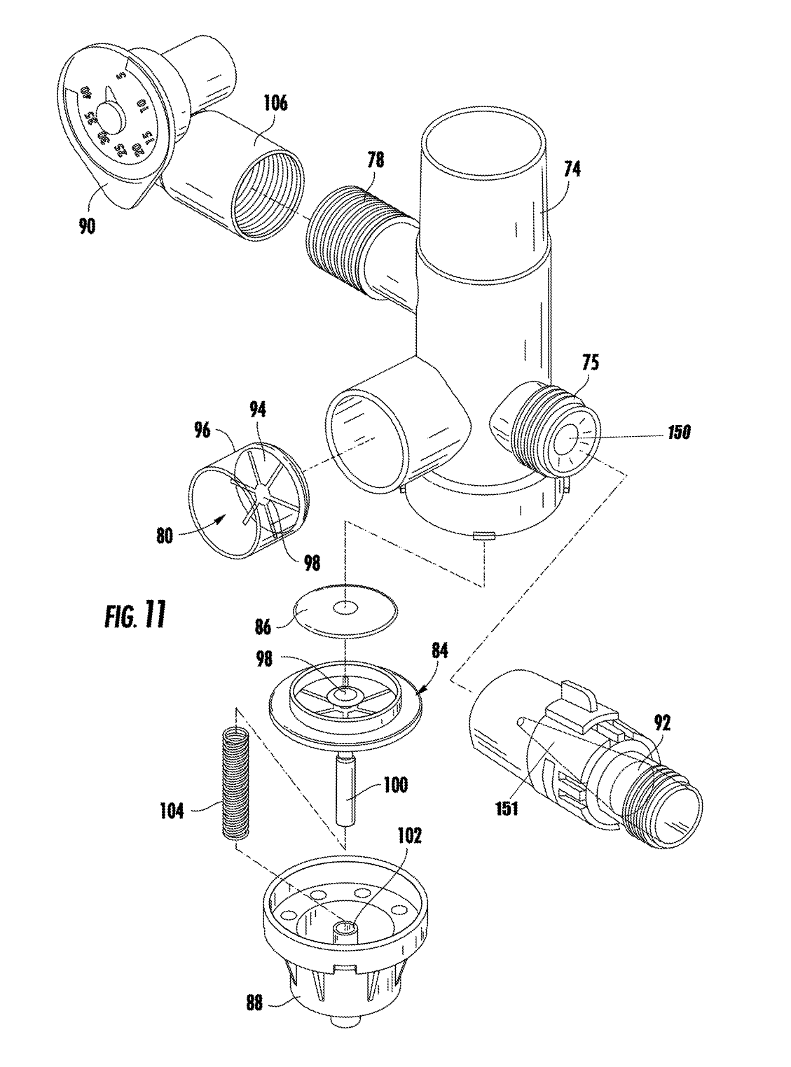

Moreover, the patient adapter 16 includes an entrainment valve 80 configured to allow additional gas flow to the patient in response to inhalation by the patient but occlude gas from escaping to the environment in response to exhalation by the patient. Thus, the entrainment valve 80 functions as a one-way valve in order to aid patients that are breathing on their own. The entrainment valve 80 may work effectively when the patient takes a quick breath, such as if the patient wakes up and panics. As shown in FIG. 11, the entrainment valve 80 includes a flexible diaphragm 94 secured to a body 96 with a fastener 98 such that the flexible diaphragm is capable of flexing inwardly in response to the patient inhaling but unable to flex outwardly in response to the patient exhaling.

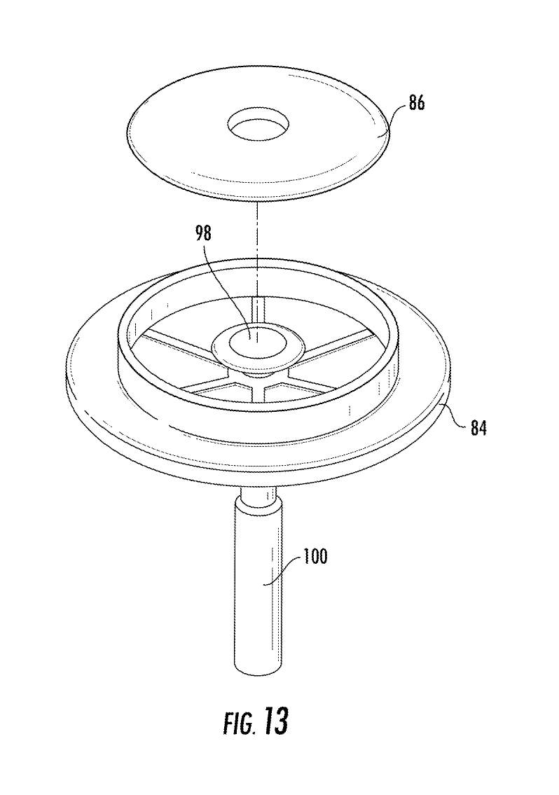

The patient adapter 16 further includes a safety pop-off valve 84 that is configured to open in response to a predetermined pressure and allow gas to escape to the environment. The safety pop-off valve 84 is surrounded by a cover 88 that is configured to prevent occlusion thereof. Namely, the cover 88 helps prevent the safety pop-off valve 84 from being occluded (e.g., a patient covering the valve) so that the functionality of the safety pop-off valve is not jeopardized. As shown in FIGS. 11 and 13, the safety pop-off valve 84 generally includes an elongate post 100 that is configured to be received within a channel 102 defined in the cover 88. A resilient member 104 such as a spring is disposed over the post 100 and the channel 102 and is configured to bias the safety pop-off valve 84 into sealing engagement with the patient adapter 16 so that air is incapable of escaping through the safety pop-off valve below a predetermined pressure (e.g., 55 cm H₂O). When the predetermined pressure within the patient adapter 16 that is sufficient to overcome the biasing force of the resilient member 104 is exceeded, the safety pop-off valve 84 opens and relieves pressure within the patient adapter.

The safety pop-off valve 84 may also include a flexible diaphragm 86 such that the safety pop-off valve also functions as an entrainment valve, and the patient is able to inhale air in addition to that provided by the entrainment valve 80, as shown in FIG. 13. Thus, below a predetermined pressure, the safety pop-off valve 84 allows the patient to inhale air therethrough but not exhale through the entrainment valve. However, if the pressure exceeds the predetermined pressure, the safety pop-off valve 84 opens. As explained above, patient entrainment of ambient air may reduce the concentration of O₂ necessary to be delivered to the patient depending on the flow rate and percentage of O₂ delivered to the gas inlet port 75. As with the entrainment valve 80, the safety pop-off valve 84 includes a fastener 98 that secures the flexible diaphragm 86 to the safety pop-off valve 84. The fastener 98 and safety pop-off valve 84 may be manufactured as one piece; however, it is preferable that the fastener 98 be manufactured separately from the safety pop-off valve to allow for the use of injection molding to manufacture these pieces. The fastener 98 can then be secured to the safety pop-off valve 84 by means of adhesive, fasteners, a snap fit, or the like. Further, the safety pop-off valve 84 can be of sufficient size and of the appropriate shape to completely replace the functionality of the entrainment valve 80. In such an embodiment, the patient adapter would include the safety pop-off valve 84 without a separate entrainment valve 80.

It is understood that the apparatus 10 may incorporate various optional features for facilitating ventilatory support and resuscitation. For example, the apparatus 10 may incorporate a heat and moisture (HME) filter that is coupled between the patient connection port 78 and the patient. The HME filter, as known to those of ordinary skill in the art, provides humidification to the patient. Moreover, the apparatus 10 may employ an ETCO₂ device to verify optimal exchanges of gases.

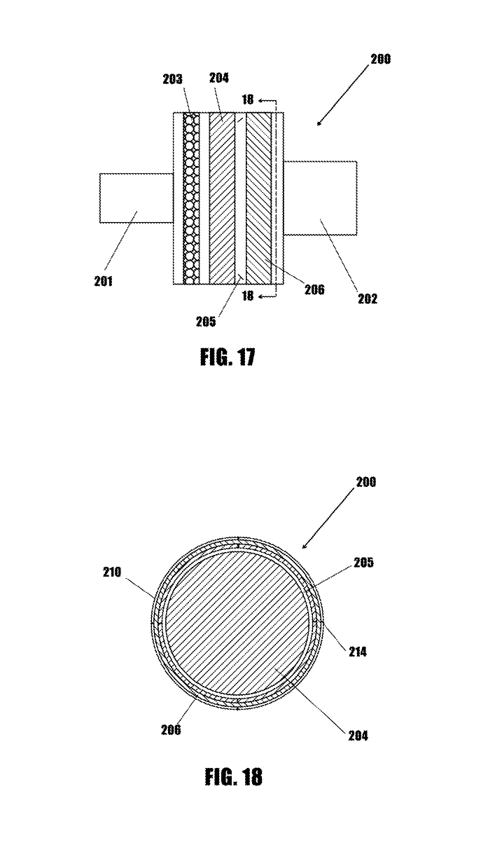

A filtration unit 200 may be coupled to the patient adapter to filter air exhaled from the patient. FIGS. 17 and 18 illustrate such a filtration unit. While air may flow in either direction, the filtration unit is intended to filter air exhaled from the patient travelling from the patient connection port 201 to the outlet port 202. Exhaled air travels through the patient connection port and into a chamber that includes various elements. First, exhaled air may travel through an optional HME filter, whereby the HME filter collects humidification from exhaled air and reintroduces humidity into air as it is inhaled by the patient. Exhaled air then travels through a HEPA filter 204, whereby 99.9% of bacteria are removed from exhaled air. Exhaled air, now purified, can pass by an optional colormetric paper that will provide a quantifiable reading of entidal CO₂. Slats, 205 connected to the main body 210 of the filtration unit 200 by means of supports 214, help direct airflow over the colormetric paper 206 thereby increasing the accuracy of the results rendered by the colormetric paper. Use of colormetric paper for rendering quantifiable readings of entidal CO₂ is known in the art, whereby the colormetric paper can indicate ranges of percent CO₂ from 0.5%-5%. To allow for sufficient airflow through the HEPA filter and optional HME filter and colormetric paper, the main body 210 of the filtration unit 200 will preferably be larger than the patient connection port 201 and the outlet port 202.

The filtration unit is preferably coupled to the patient connection port 78, whereby the outlet port 202 of the filtration unit is proximate to the patient connection port 78. Any components downstream of the filtration unit, meaning exhaled air passes through the filtration unit before passing through that component, may be reused without sterilization. Thus, by using the filtration unit coupled directly or indirectly to the patient connection port 78, various components, including the patient adapter 16 and modulator 14, can be reused between multiple patients. The filtration unit can also be coupled to the inlet port 22 and outlet port 74, whereby the outlet port 202 of the filtration unit couples to the inlet port 22 and the patient connection port 201 couples to the outlet port 74. In this fashion, the modulator 14 encounters filtered air and may be reused for multiple patients, whereas the patient adapter encounters unfiltered exhaled air from the patient and should be disposed of after use by a single patient.

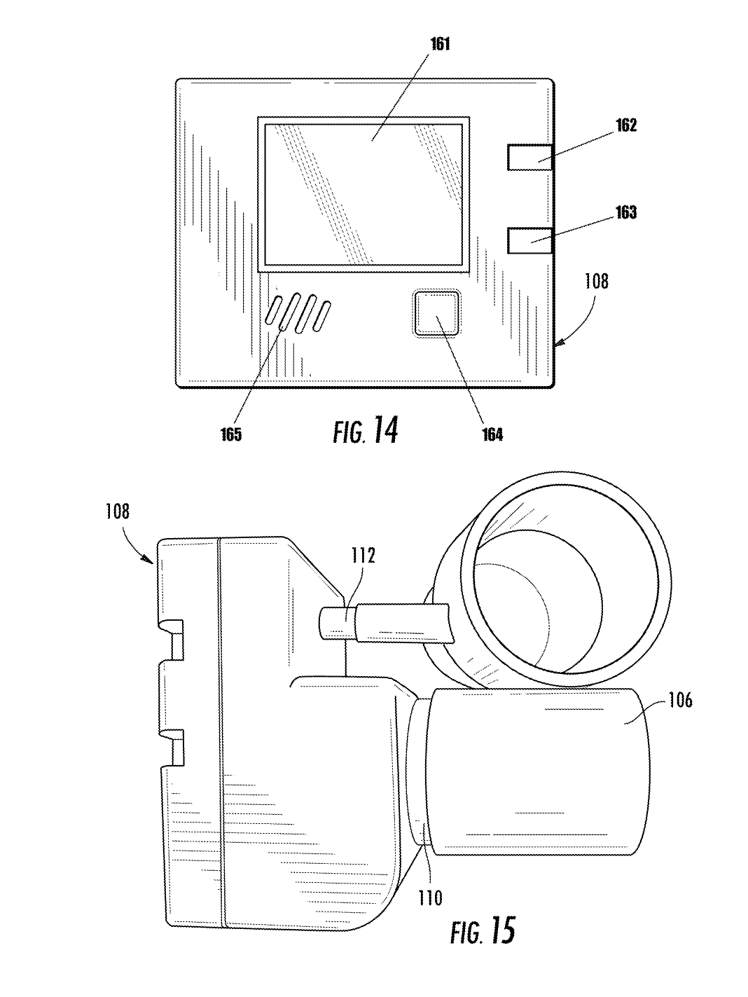

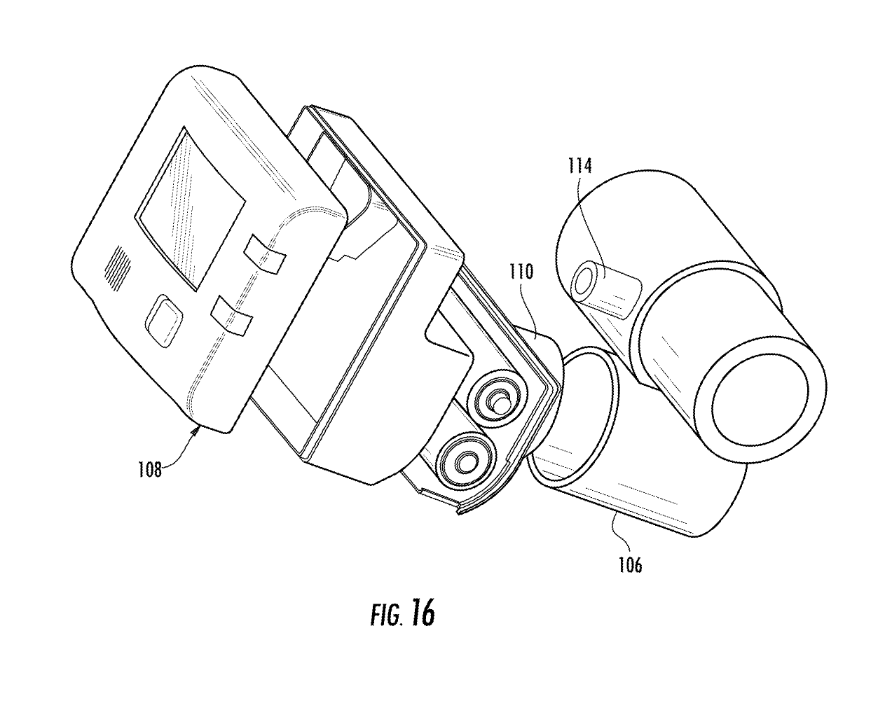

Furthermore, FIGS. 1, 10, and 11 illustrate that a manometer 90 may be coupled to the patient connection port 78 with a coupling 106 in order to monitor the pressure flowing through the patient adapter 16. Alternatively, the manometer 90, 108 could be connected between the modulator 14 and the patient adapter 16. The manometer 90 may be a mechanical gauge for measuring pressure in cm H₂O. Alternatively, FIGS. 14-16 illustrate that the manometer 108 may be electronic (e.g., battery powered) and equipped with additional features for monitoring the patient, such as measuring rate and pressure. In addition, the manometer 108 may include an audible and/or a visual alarm for signaling various events, such as when a predetermined pressure/rate is reached. Each manometer 90, 108 may be disposable and, thus, be used for single patient use. In addition, each manometer 90, 108 may include a connector 110 that is configured to mate with the coupling 106 and a stem 112 having an opening that is configured to receive air from a corresponding stem 114 extending from the coupling.

The electronic manometer, as illustrated in FIGS. 14-16, provide significant benefits over a mechanical gauge. In one embodiment, the manometer 108 includes a screen to display information to a user, such as pressure and/or breathing rate of the patient. A speaker provides a means of producing an audible alarm, whereby the alarm sounds after a preset condition is reached or exceeded. For example, if the measured pressure falls below 10 cm H₂O, an alarm may sound. Alternatively or in addition to the audible alarm produced by the speaker, a visual alarm may be displayed. A light, preferably a light emitting diode, is displayed after a preset condition is reached or exceeded. Optionally, an additional light may be included to notify the user when the batteries of the manometer 108 are low. An audible alarm may also be used to indicate a low battery warning. While multiple buttons may be used, a single button is preferably included with the manometer. The single button can be used to power the device on, and power the device off. In this manner, there is a lesser chance of a user hitting the wrong button or inputting incorrect settings. A pressure sensor, such as one provided by Motorola, can be connected to a standard PC board programmed to activate the audible and/or visual alarms as well as to drive the information displayed on the screen.

The electronic manometer 108 should be able to withstand substantial inundation of water. To further this goal, the casing of the manometer 108 should be ultrasonically welded. While openings for a speaker may be included in the casing, a protective seal, such as a sticker, should be placed over the manometer to help prevent moisture from entering the unit. An opening may be left for the button, but the button should be sealed to prevent water from entering the manometer. By manufacturing such an embodiment as described above, an IP44 rating can be achieved. The manometer 108 is battery powered, preferably by using two AAA sized batteries. This provides a sufficient voltage to power the manometer as well as providing a battery life that exceeds 120 hours of operation.

The electronic manometer, while providing additional useful features, is more expensive to manufacture. Therefore, it is desirable to be able to reuse the manometer with multiple different patients over an extended period of time. However, if the manometer encounters unfiltered air exhaled from a patient, it must be disposed of or properly disinfected. However, by using the filtration unit disclosed above, where the filtration unit filters exhaled air passing therethrough before reaching the manometer, the manometer may be reused among several patients. Thus, it is preferable to couple the manometer to the outlet port 202 of the filtration unit and the patient connection port 78 of the patient adapter 16. Alternatively, the manometer may be coupled to the patient connection port 78 and the inlet port 22. In fact, there are multiple configurations of coupling the manometer to the patient adapter 16 and/or modulator 14 downstream from the filtration unit 200 that will be appreciated by one skilled in the art.

In use, the apparatus 10 is typically used for patients (e.g., patients weighing over 8 kilograms or 18 lbs) who are in need of emergency, short term, pressure cycled ventilatory support and for those patients unable to maintain an adequate blood gas (PH) status during unassisted ventilation. The apparatus 10 is configured to adjust the flow rate, peak inspiratory pressure (PIP), and respiratory rate for providing proper ventilatory support and resuscitation to the patient. In particular, the flow rate may be adjusted by turning the flow rate dial 28 to maintain a desired rate. The respiratory rate may be adjusted by the amount of inspiratory flow of O₂ delivered to the patient through the inlet port 75. Moreover, the PIP may be adjusted by turning the pressure dial 26 to a desired pressure range. Typically, adjustment of the PIP also requires adjustment of the flow rate in order to achieve pressure supported ventilatory support. In addition, the higher the flow rate is, the shorter the inspiration time will be, and the lower the flow rate is, the longer the inspiration time will be. Thus, adjustment of the flow rate, PIP, and respiratory rate ensures that the automatic pressure cycled ventilatory support is provided to each patient.

The apparatus 10 is pressure cycled on PIP as well as positive end expiratory pressure (PEEP), wherein PEEP is typically about 25% of PIP. Thus, the apparatus 10 is pressure cycled on inhalation and exhalation (PIP and PEEP), which may minimize the possibility of gas trapping. During inhalation, the exhalation phase is not activated until PIP is reached. During exhalation, the inhalation phase will not commence until the pressure drops to PEEP. For the spontaneously breathing patient the pressure setting (i.e., positive airway pressure (PAP)) is set above the intrinsic PEEP allowing the patient to initiate inhalation phase by drawing down to the set PEEP. When using PAP, the airway pressure is above the intrinsic PEEP setting. For example, for PIP set at 25 cm H₂O, PEEP is 6 cm H₂O, and the PAP setting needs to be 7 cm H₂O or higher. In addition, if the orifice 50 of the flow restrictor 42 is variable as described above, the PEEP setting may be infinitely adjustable for maintaining a constant airway pressure when in the PAP mode 5-30+cm H₂O.

The apparatus 10 is supplied with an appropriate source of gas flow (inspiratory flow) and operates on a continuous inspiratory flow. For example, supply pressures from 1 BAR (15 PSI) to 5 BAR (75 PSI) may be used so long as an inspiratory flow of adjusted 10 to 40 liters per min (LPM) is used. For flow and rate control, a flow meter capable of settings to 40 LPM or alternately 0-25 LPM with flush (flush provides 40 LPM) may be used, but the apparatus 10 may be connected directly to a 3.5 BAR (50 PSI) gas source. According to one embodiment, the apparatus 10 is designed to automatically deliver 30+LPM of O₂ when connected directly to a 3.5 BAR (50 PSI) gas source. The flow rate needed for most patients is typically about 15-20 LPM.

When using an orifice-type flow regulator, commonly used on most cylinder tanks, the amount of flow delivered will be indicated by the regulator setting. If the regulator being used has a high flow port connection and the apparatus 10 is connected to this port, flow may be 30+LPM if the regulator is set to 3.5 BAR (50 PSI). The apparatus 10 may deliver 100% O₂ to a patient when the apparatus is supplied with 100% O₂. If a lower O₂ concentration is desired, an oxygen blender at the gas source (wall outlet or regulator outlet) may be used. As discussed above, an entrainment unit 92 may be employed to reduce the amount of O₂ delivered to the patient. For example, the following table provides exemplary flow rate values when using an entrainment unit 92 in an open position (e.g., 6 LPM of 100% O₂ is delivered for a total delivered flow rate of 20 LPM):

TABLE 1
Entrained Mode Flow Chart (LPM)

| | | | | | |
|---|---|---|---|---|---|
| 100% O₂ Input | 6 | 8 | 10 | 12 | 15 |
| Delivered at 65% O₂ | 12 | 16 | 20 | 24 | 30 |

Commonly used therapy entrainment devices are typically not recommended for this purpose as they most often will not provide the flow or pressure required to properly operate the apparatus 10.

In use, a supply tubing is connected to the apparatus 10 and to an appropriate supply source of gas flow or inspiratory flow as described above. For example, the tubing may be connected to the inlet port 75 (e.g., with a DISS fitting) and then to the oxygen source (e.g., with a DISS fitting or push-on barb fitting). The apparatus 10 may be pre-set at a predetermined PIP and inspiratory flow. For instance, the initial settings may be a PIP of about 25 cm H₂O and an inspiratory flow of about 15 LPM, which corresponds to a rate of about 12-14 BPM (breaths per minute). If adjustment to the PIP is desired, the pressure dial 26 may be rotated to the desired setting, such as between 10 to 40 cm H₂O. When ventilating an intubated patient, higher PIP settings may be required. Once the flow and PIP have been set, a function check should be performed on the apparatus 10 before connecting it to the patient. This check may be accomplished by occluding the patient connection port 78 and verifying that the modulator opens and the pressure does not exceed a predetermined pressure, such as about 60 cm H₂O.

The patient is then connected to the apparatus 10, such as with an endotracheal tube. The endotracheal tube may be connected directly to the patient adapter 16 and HME. If a face mask is employed, the mouth and airway are typically cleared of foreign bodies and conventional techniques are used to ensure correct position of the airway. The mask is typically held firmly against the face while keeping the head properly positioned. If the patient vomits, the patient adapter 16 should be disconnected from the modulator 14 and the rate dial should also be removed if necessary for clearing. Once the vomit has been removed, the modulator 14 and patient adapter may be reconnected. Depending on the patient and circumstances, the clearing procedure may be accomplished rather quickly, such as in less than 20 seconds. Once the patient's airway is clear, ventilation may be resumed and monitored to ensure that inhalation and exhalation occur without obstruction.

The flow rate may be adjusted to achieve the desired respiratory rate after the patient is connected to the apparatus 10. For example, the flow rate may be adjusted to achieve anywhere between 8 and 30 BPM, and 12 BPM according to one embodiment. The flow rate may be adjusted by turning the flow rate dial 28 to increase or decrease the flow rate, such as in ¼ turn increments. The flow rate may be decreased by turning the flow rate dial 28 clockwise or increased by turning the flow rate dial counterclockwise. If the desired decrease in flow rate cannot be attained, an increase in inspiratory flow is most likely necessary. For example, if the current flow is 15 LPM, the new flow should be increased to 20+LPM.

Delivered tidal volume (i.e., the normal amount of air exchanged between inhalation and exhalation) may be determined by multiplying the gas flow in ml/second and the inspiratory time in seconds, wherein the inspiratory time may be determined by counting the seconds between when inspiration begins and ends, or by using the following tidal volume estimator table.

TABLE 2
Tidal Volume Estimator

INSPIRATORY RATE (in seconds)

| | | | | | | |
|---|---|---|---|---|---|---|
| FLOW | 0.5 | 1 | 1.5 | 2 | 2.5 | 3 |

| | |
|---|---|
| (LPM) | Tidal Volume (ml) |

| | | | | | | |
|---|---|---|---|---|---|---|
| 15 | 125 | 250 | 375 | 500 | 625 | 750 |
| 20 | 167 | 333 | 500 | 667 | 833 | 1000 |
| 25 | 208 | 417 | 625 | 833 | 1042 | 1250 |
| 30 | 250 | 500 | 770 | 1000 | 1250 | 1500 |
| 35 | 292 | 583 | 875 | 1167 | 1458 | 1750 |
| 40 | 333 | 667 | 1000 | 1333 | 1667 | 2000 |

For a breathing patient who needs ventilatory support, the rate may be adjusted such that the patient triggers a breath. In particular, for breathing patients, the rate dial 28 may control exhalation time (texhale) and when set low enough, will cause the modulator 14 to stop cycling automatically thereby delivering pressure supported ventilatory support (PAP), which the patient must trigger through inspiration effort to begin subsequent full inhalations. For instance, rate may be adjusted by turning the rate dial 28 clockwise until the modulator stops cycling and the desired pressure is reached (e.g., 5-10 cm H₂O). In the PAP mode, exhalation time is determined by the patient.

For non-breathing patients (e.g., if the patient is experiencing apnea) or if pressure controlled ventilation is otherwise desired, automatic cycling of the modulator 14 can be initiated by adjusting the rate dial 28 to increase the flow rate until cycling begins. The rate dial 28 may be adjusted to the desired rate, and the breathing rate may be determined manually (e.g., such as by counting 1-1000, 2-1000, etc.) or with a timing device. In the pressure control mode, there is no prolonged stage where the flow of exhalation gas stops for a significant duration of time. This occurs because the exhalation time is set with the rate dial 28 by varying the exhalation resistance so that the patient just finishes exhalation with the beginning of the subsequent inhalation.

Other techniques used to ensure proper respiratory rate include observing the rise and fall of the patient's chest corresponding to inhalation and exhalation of the patient, listening for expiratory flow from the modulator 14 (e.g., coming from the rate dial 28), and listening to breath sounds of the patient. The flow rate may also need to be adjusted if PIP is adjusted, which may be controlled by rotating the rate dial 28 as necessary. Although the pressure dial 26 may have markings indicating an approximate pressure, the settings may be verified with a manometer 90, 108. Moreover, changes in the patient's lung compliance may result in respiratory rate changes such that appropriate adjustments of rate and pressure may be necessary. It is recommended that the apparatus 10 be used only by individuals that are trained to do so, including those with training in CPR and the operation of gas-powered resuscitators.

Embodiments of the present invention may provide several advantages. For example, the apparatus 10 may provide consistent, reliable, and hands-free ventilatory support and resuscitation. In particular, the apparatus 10 may provide short-term ventilatory support (e.g., less than 7 days), as well as constant flow, pressure cycled ventilatory support for breathing and non-breathing patients. The apparatus 10 is also entirely gas driven such that no external power source is necessary. In addition, the apparatus 10 may be indicated for single patient use such that dis-assembly, cleaning, sterilization, and maintenance are not required. For example, most if not all of the components of the modulator 14 and patient adapter 16 may be manufactured from polymeric materials and formed using conventional techniques such as injection molding. Moreover, the apparatus 10 includes safeguards for ensuring the patients safety, such as a safety-pop off valve, pressure control settings, and audible inhalation and exhalation detections that are recognizable during operation of the apparatus.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

That which is claimed:

1. An apparatus for providing ventilatory support and resuscitation for a patient, the apparatus comprising:
a chamber;
at least one port in flow communication with the chamber; and
a flexible diaphragm member positioned within the chamber and configured to seal the port from flow communication with the chamber while seated adjacent to the port and to flex in response to pressure applied thereto so as to allow flow communication between the port and the chamber while the diaphragm is flexing.

2. The apparatus of claim 1, wherein the diaphragm comprises an inner region and an outer region, and wherein the inner region is configured to seal the port and the outer region is configured to flex in response to pressure applied to the inner region

3. The apparatus of claim 2, wherein the diaphragm comprises an outer edge, and wherein the outer edge is configured to frictionally engage the chamber and remain stationary while the outer region is flexing.

4. The apparatus of claim 2, wherein the inner region comprises a more rigid material than the outer region.

5. The apparatus of claim 2, wherein the outer region comprises a plurality of annular ridges, and wherein the outer region is configured to flex along the annular ridges.

6. The apparatus of claim 1, further comprising a resilient member configured to apply a biasing force against the diaphragm so as to bias the diaphragm adjacent to the port, wherein the diaphragm is configured to flex so as to overcome the biasing force in response to pressure applied thereto.

7. The apparatus of claim 6, further comprising a dial for selectively adjusting the pressure required to overcome the biasing force of the resilient member.

8. The apparatus of claim 1, further comprising a flow restrictor in flow communication with the chamber, wherein the flow restrictor is configured to be in flow communication with the port when the diaphragm is flexing.

9. The apparatus of claim 8, wherein the flow restrictor has a proximal end and a distal end, and wherein the flow restrictor comprises an orifice extending through and between the proximal and distal ends for facilitating flow communication between the chamber and the environment.

10. The apparatus of claim 1, further comprising a patient adapter coupled to the port and in flow communication therewith.

11. The apparatus of claim 10, wherein the patient adapter further comprises an inlet port configured to couple with tubing supplying a gas.

12. The apparatus of claim 11, further comprising an entrainment unit, where the entrainment unit comprises an opening, where the entrainment unit couples the inlet port with the tubing supplying a gas, where the opening allows ambient air to enter the inlet port.

13. The apparatus of claim 10, further comprising a manometer coupled to the patient adapter.

14. The apparatus of claim 13, wherein the manometer is an electronic manometer, where the electronic manometer comprises a screen, a speaker, and a light.

15. The apparatus of claim 13, wherein the manometer is an electronic manometer, where the electronic manometer comprises only 1 button.

16. The apparatus of claim 13, wherein the manometer is an electronic manometer, where the electronic manometer comprises an adhesive sticker.

17. The apparatus of claim 10, wherein the patient adapter further comprises at least one entrainment valve configured to allow additional gas flow to the patient in response to inhalation by the patient but to occlude gas from escaping to the environment in response to exhalation by the patient.

18. The apparatus of claim 17, wherein the entrainment valve comprises a safety pop-off valve configured to open in response to a predetermined pressure and allow gas to escape to the environment.

19. The apparatus of claim 18, further comprising a cover surrounding the safety pop-off valve and configured to prevent occlusion thereof.

20. The apparatus of claim 10, further comprising a filtration unit, where the filtration unit comprises a high efficiency particulate absorbing filter.

21. The apparatus of claim 20, wherein the filtration unit further comprises a heat and moisture filter.

22. The apparatus of claim 20, wherein the filtration unit further comprises colormetric paper.

23. A method for providing ventilatory support and resuscitation for a patient, the method comprising:
providing an apparatus comprising:
a chamber;
at least one port in flow communication with the chamber; and
a flexible diaphragm member positioned within the chamber and configured to seal the port from flow communication with the chamber while seated adjacent to the port; and
coupling the apparatus to a patient such that the patient and apparatus are in flow communication with one another and such that the diaphragm is configured to flex in response to pressure applied thereto resulting from exhalation by the patient so as to allow flow communication between the port and the chamber while the diaphragm is flexing.

24. The method of claim 23, further comprising selectively adjusting the pressure required to flex the diaphragm.

25. The method of claim 23, further comprising selectively adjusting the flow rate of gas exiting the chamber.

26. The method of claim 23, further comprising coupling a patient adapter to the port, wherein the patient adapter is configured to receive a gas source and deliver the gas source to the patient.

27. A patient adapter configured to be coupled with an apparatus for providing ventilatory support and resuscitation for a patient, the patient adapter comprising:
an inlet port configured to couple with a tubing supplying a gas;
a patient connection port configured to deliver gas to the patient; and
at least one entrainment valve configured to allow additional gas flow to the patient in response to inhalation by the patient but occlude gas from escaping to the environment in response to exhalation by the patient, wherein the at least one entrainment valve comprises a safety pop-off valve configured to open in response to a predetermined pressure and allow gas to escape to the environment.

28. The patient adapter of claim 27, further comprising a cover surrounding the safety pop-off valve and configured to prevent occlusion thereof.

29. The patient adapter of claim 27, further comprising an entrainment unit, where the entrainment unit comprises an opening, where the entrainment unit couples the inlet port with the tubing supplying a gas, where the opening allows ambient air to enter the inlet port.
